(19) United States
(12) Patent Application Publication
(10) Pub. No.: US 2026/0077197 A1
(43) Pub. Date: Mar. 19, 2026

(54) SYSTEMS AND METHODS FOR OCULAR MICROSTIMULATION THERAPY

(71) Applicant: i-Lumen Scientific, Inc., Bloomington, MN (US)

(72) Inventors: Conor M. Minogue, Kinvara (IE); Meredith Mundy, Versailles, KY (US); John C. VeLure, Minnetonka, MN (US); Marshall T. Masko, Minnetonka, MN (US)

(21) Appl. No.: 19/043,281

(22) Filed: Jan. 31, 2025

Related U.S. Application Data
(60) Provisional application No. 63/548,804, filed on Feb. 1, 2024.

(51) Int. Cl. A61N 1/36; A61N 1/04

(52) U.S. Cl. CPC A61N 1/36046; A61N 1/0408; A61N 1/0472; A61N 1/36025; A61N 1/36034

(57) ABSTRACT

A system and method for delivering ocular microstimulation therapy includes one or more active electrodes near the anterior part of the eye, one or more return electrodes on the patient, and a controller configured to generate electrical signals (e.g., anodal monophasic or biphasic pulses) with defined amplitude, current density, polarity, frequency, and duty cycle. The total electrode contact area is between about 1.5 cm² and about 4 cm². In some methods, the stimulation can be divided into subphases or varied in ramped or randomized patterns to reduce habituation. A therapy regimen can span multiple sessions, with data structures specifying session dates and modes, and whether treatment is unilateral or bilateral. Safety features detect hazardous reductions in electrode contact area.

### CROSS-REFERENCE TO RELATED APPLICATIONS

This application claims the benefit of U.S. Provisional Ser. No. 63/548,804, filed on Feb. 1, 2024, the entire contents of which are incorporated herein by reference in their entirety.

This application is related to:
U.S. Pat. No. 10,391,312, issued on Aug. 27, 2019 by Mowery et al., titled “APPARATUS AND METHOD FOR OCULAR MICROCURRENT STIMULATION THERAPY,”
PCT Patent Application PCT/US2016/051550 (published as WO 2017/048731), filed Sep. 13, 2016 by Mowery et al., titled “APPARATUS AND METHOD FOR OCULAR MICROCURRENT STIMULATION THERAPY,”
U.S. Provisional Patent Application 62/283,870, filed Sep. 15, 2015 by Mowery et al., titled “APPLIANCE FOR MICROCURRENT STIMULATION THERAPY USING A DISPOSABLE MATERIAL AFIXED TO THE UPPER AND LOWER EYE LID & OTHER BODY PARTS,”
U.S. Provisional Patent Application 62/283,871, filed Sep. 15, 2015 by Masko et al., titled “APPARATUS FOR A METHOD OF APPLICATION OF MICROCURRENT STIMULATION THERAPY, CONSISTING OF A GOGGLE DEVICE AFFIXED TO & ENCIRCLING THE UPPER AND/OR LOWER EYELIDS, AS WELL AS OTHER BODY PARTS,”
U.S. Provisional Patent Application 62/365,838, filed Jul. 22, 2016 by Tapp et al., titled “APPLIANCE FOR MICRO-CURRENT STIMULATION,”
U.S. patent application Ser. No. 17/415,508 (published as US 2022/0047866), filed Jun. 17, 2021 by Masko et al., titled “APPARATUS AND METHOD FOR MICROCURRENT STIMULATION THERAPY,”
U.S. patent application Ser. No. 17/416,024, filed Jun. 18, 2021 by Masko et al., titled “MICROCURRENT-STIMULATION-THERAPY APPARATUS AND METHOD” (issued Sep. 13, 2022 as U.S. Pat. No.11,439,823),
PCT Patent Application PCT/US2019/063404 (published as WO 2020/131329), filed on Nov. 26, 2019, by Masko et al., titled “APPARATUS AND METHOD FOR MICROCURRENT STIMULATION THERAPY,”
PCT Application PCT/US2019/067627 (published as WO 2020/132337), filed on Dec. 19, 2019 by Masko et al., titled “MICROCURRENT-STIMULATION-THERAPY APPARATUS AND METHOD,”
U.S. Provisional Patent Application 62/783,116 filed on Dec. 20, 2018 by Masko et al., titled “APPARATUS AND METHOD FOR MICROCURRENT STIMULATION THERAPY,”
PCT Patent Application PCT/US2020/021267 (published as WO 2021/177968), filed Mar. 5, 2020 by Mowery et al. titled “VISION TESTING AND TREATMENT SYSTEM AND METHOD,”
PCT Patent Application PCT/US2021/031869 (published as WO 2021/231496), filed May 11, 2021 by Duncan et al. titled “ELECTRODE SYSTEM FOR VISION TREATMENT AND METHOD,”
U.S. Provisional Patent Application 63/025,987 filed on May 15, 2020 by Duncan et al., titled “ELECTRODE SYSTEM FOR VISION TREATMENT AND METHOD,”
U.S. Pat. No. 11,116,973, issued Sep. 14, 2021 to Masko et al., titled “SYSTEM AND METHOD FOR A MEDICAL DEVICE,” and
U.S. Provisional Patent Application 63/281,558 filed on Nov. 19, 2021 by Masko et al., titled “METHOD AND SYSTEM FOR EYE TREATMENT,”
PCT Application PCT/US2022/050319 (published as WO 2023091611), filed on Nov. 17, 2022, by Masko et al., titled “METHOD AND SYSTEM FOR EYE TREATMENT,” each of which is incorporated herein by reference in its entirety.

### TECHNICAL FIELD

This disclosure relates to the treatment of vision disorders (e.g., ocular disease), more particularly, the selective therapeutic application of energy, in the form of electrical energy, to the eye(s).

### BACKGROUND

The most common causes of vision impairment among adults in the United States are age-related macular degeneration (AMD), geographic atrophy, presbyopia, diabetic retinopathy, cataracts, dry eye disease (DED), and glaucoma.

AMD is a degenerative, progressive disease that affects the part of the retina that is responsible for sharp central vision and is the leading cause of legal blindness/visual impairment in the United States in persons over 50 years old. AMD begins as the dry form (non-exudative AMD) with the development of extracellular deposits called drusen in between the Bruch's membrane and the retinal pigment epithelial (RPE) cells. Drusen deposits are composed of various proteins and lipids and the size/number of these deposits provides a classification for the stage of AMD with more advanced forms having more and larger deposits. While research in this area over several decades has shown promising benefits to patients with dry AMD, there are currently no FDA cleared or approved treatments in the U.S. to prevent, slow or restore vision loss for patients with dry AMD.

Advanced AMD referred to as geographic atrophy affects the outer neural layer (photoreceptors and neurons), the RPE cell layer adjacent to Bruch's membrane, and the choriocapillaris of the macula, and eventually leads to irreversible vision loss. In GA, because the RPE regulates the transmission of nutrients from the choriocapillaris in the choroid, disruption to this interface results in reduced perfusion to the retina and leads to reduced nutrient availability, metabolic stress, and starvation for the RPE and photoreceptors, which ultimately leads to atrophy of the retinal tissue in that area and clearly demarcated loss of RPE. On the other hand, neovascular AMD is characterized by the formation of new blood vessels from the choroid that extend into and through the retinal layers. These new blood vessels are highly permeable and lead to leakage of fluid and additional deposition into and below the retina, which leads to scarring, increased RPE detachment, and ultimately cell death.

Normal retinal cell function is a photochemical reaction converting light energy to an electrical impulse which travels to the brain and vision occurs. With AMD and other visual system diseases, diseased, inflamed retinal cells eventually lose cell function. Adenosine triphosphate (ATP) levels drop, protein synthesis drops, the electrical resistance goes up, and cell electricity potential goes down. Basically, the cells seem to go dormant for a time before they die. It is believed that, if electrical stimulation is provided to the cells before they die, blood vessel permeability is increased, a more normal cellular electrical potential will be achieved, the ATP levels will increase, protein synthesis will occur again, and normal cell metabolism will be restored.

However, there remains a need for an energy-based treatment of vision disorders that maximizes therapeutic effect.

### SUMMARY OF INVENTIONS

In some embodiments, methods of using an ocular microstimulation system comprising one or more stimulation electrode assemblies and a controlled are disclosed. The controller comprises a programmable memory storing a plurality of patterned stimulation waveforms, the method comprising applying a patterned electrical stimulation comprising a first biphasic waveform of the plurality of patterned stimulation waveforms transpalpebrally to a patient's first eye, and applying a patterned electrical stimulation comprising a monophasic waveform or a second biphasic waveform of the plurality of patterned stimulation waveforms transpalpebrally to a patient's second eye simultaneously. The biphasic waveform comprises a plurality of first phase pulses, each of the first phase pulses, and a plurality of second phase pulses having an opposite polarity of the first phase pulses.

In some embodiments, a system for delivering electrical stimulation to at least one eye of a patient comprises: one or more active electrodes configured to be positioned near the anterior part of the eye; one or more return electrodes configured to be positioned on the patient; and a controller operably coupled to the one or more active electrodes and the one or more return electrodes, the controller being configured to generate electrical stimulation signals having a defined amplitude, current density, polarity, frequency, and duty cycle, wherein the electrical stimulation signals, including anodal monophasic pulses, are delivered transpalpebrally or via direct corneal contact through the one or more active electrodes having a total area of conductive contact of between about 1.5 cm² and about 4 cm².

The system may provide stimulation that comprises two or more phases, at least one of which delivers monophasic pulses and at least one of which delivers biphasic pulses, and the amplitude of the monophasic pulses may be different from the amplitude of the biphasic pulses.

A monophasic phase may be divided into a plurality of subphases, each subphase delivering a train of pulses having exclusively positive polarity or exclusively negative polarity.

The subphases may be arranged to deliver an equal accumulated electrical charge and/or to limit the total accumulated electrical charge in each subphase to less than a predefined threshold.

Each subphase may have a duration of at least about 1 second, optionally about 5 seconds, 10 seconds, 30 seconds, or 60 seconds.

An accumulated charge in at least the monophasic phase may be controlled by adjusting either the amplitude or the pulse width of the pulses.

The frequency of the monophasic pulses may be between about 20 Hz and about 40 Hz.

The amplitudes of the monophasic and biphasic phases may be determined based on the patient's tolerance threshold for each mode and may be automatically managed thereafter.

The frequency of the biphasic phase may be in the range of about 15 Hz to about 25 Hz.

The two or more phases may be separated by a rest phase in which no stimulation is provided.

The duration of a phase, whether monophasic, biphasic, or rest, may be in the range of about 0.1 to 10 minutes, preferably about 1 minute.

A treatment session may comprise delivering the monophasic phase and the biphasic phase in either order.

The respective durations of the monophasic and biphasic phases may be unequal.

A treatment regimen may comprise multiple treatment sessions in which the proportions of the monophasic and biphasic phases are varied according to a defined schedule.

The controller may be further configured to implement a therapy regimen for the patient that spans multiple treatment sessions, each session being exclusively monophasic, exclusively biphasic, or a combination of monophasic and biphasic pulses, the regimen being defined by a therapy calendar specifying dates and the stimulation parameters for each session.

The therapy calendar may be defined by a data structure that includes a patient identifier, a total number of treatment sessions, and dates of treatment.

The data structure may further specify, for each treatment session, whether the eye treatment is unilateral, sequential bilateral, or simultaneous bilateral.

For each eye treated in each session, the data structure may specify a session duration and a pulse train description including a number of phases.

For each phase of the session, the data structure may specify whether the phase is monophasic or biphasic, a frequency, an amplitude, a pulse width, a burst length, and a polarity.

The one or more active electrodes may be located near the anterior part of the eye, and the one or more return electrodes may be located posterior to the eye.

The one or more return electrodes may have a conductive contact area that is at least twice that of the one or more active electrodes.

The controller may be configured to limit the stimulation signal so that the direct current (DC) density at the one or more active electrodes does not exceed about 0.5 mA/cm² or more preferably, 0.25 mA/cm².

The controller may be configured to limit the time-integrated charge density at the one or more active electrodes to less than a predefined limit.

The controller may include a mechanism to detect a potentially hazardous reduction in the contact area of the one or more active electrodes.

The mechanism may comprise detecting an increase in an area-dependent electrical impedance parameter beyond an acceptance threshold.

The acceptance threshold may be determined during a calibration phase in which the one or more active electrodes are known to be fresh and fully contacting, and by setting it to a predefined amount higher than the parameter measured during the calibration phase.

A switching mechanism may be further provided, operably connected to the controller and configured to direct stimulation pulses bilaterally to both eyes of a patient, such that each eye receives its own pulse train having defined polarity, amplitude, frequency, or duty cycle.

Pulses may be time-multiplexed to both eyes.

The controller may deliver bursts of pulses alternately to each eye such that, while one eye is receiving pulses, the other eye is resting.

The two eyes may receive differing stimulation parameters, including any one or more of monophasic vs. biphasic waveforms, amplitude, frequency, or pulse width.

The controller may be configured to generate a periodic pulse train having net zero DC current, in which positive and negative parts of the pulse train are asymmetric in amplitude or duration while maintaining overall charge balance.

The asymmetric waveform may comprise positive and negative rectangular, exponential, or triangular pulses of equal area but different duration.

The asymmetric waveform may comprise a sawtooth shape in which a positive slope extends for a longer duration than a negative slope.

The positive slope may refer to a positive rate of change of current entering the one or more active electrodes.

The controller may be configured to periodically vary at least one stimulation parameter selected from amplitude, pulse width, or frequency in a ramped or randomized manner over time, thereby reducing patient habituation during ocular stimulation.

In some embodiments, when an average DC current density at the one or more active electrode is about 50 to 500 μA/cm², an average DC current density at a posterior portion of a retina of the eye is 5 to 50 μA/cm².

### BRIEF DESCRIPTION OF THE DRAWINGS

FIG. 1 is a block diagram illustrating the system for providing electrical stimulation to a patient's eye in accordance with one more embodiments of the invention.

FIG. 2 is a map of the eye showing the outward radial flow of current (indicated by arrow vectors) and highlighting the current density at the posterior portion of the retina.

FIG. 3A illustrates an example pulse train comprising alternating monophasic pulse train segments having opposite polarity in accordance with one more embodiments of the invention.

FIG. 3B illustrates an example pulse train comprising monophasic pulse train segments with an inactive or rest segment in accordance with one more embodiments of the invention.

FIG. 3C illustrates an example bilateral stimulation wherein pulses are time multiplexed between each eye (electrode C may serve as a common return such that biphasic or monophasic pulses can be delivered without interaction between the eyes) in accordance with one more embodiments of the invention.

FIG. 3D illustrates example bilateral multiplexed biphasic pulses in accordance with one more embodiments of the invention.

FIG. 3E illustrates an example bilateral multiplexed pulse trains with monophasic segments of opposite polarity in accordance with one more embodiments of the invention.

FIG. 3F illustrates example asymmetric biphasic waveforms of the same frequency f=1/T Hz, but with different phase durations and slope. Both waveforms are charge balanced with zero net DC in accordance with one more embodiments of the invention.

FIG. 3G illustrates an example asymmetric balanced biphasic pulse, showing a leading phase of amplitude V1 for a time t1 with a second phase of longer duration, t2, of lesser amplitude V2. The pulse is repeated after time T but, in this example, the leading phase of the second pulse is of opposite polarity to the preceding pulse. In some embodiments, a sequence of pulses would have the same leading phase polarity in accordance with one more embodiments of the invention.

FIG. 3H illustrates an example asymmetric biphasic current waveform (top) which, despite a charge balance between phases, is biased through having a more sustained positive rate of change of current, di/dt, than negative (bottom) in accordance with one more embodiments of the invention.

FIG. 4 illustrates an example computing device in accordance with one more embodiments of the invention.

FIG. 5A illustrates maintenance treatment regimens (Mono/Bi) over daily and monthly intervals across various studies.

FIG. 5B illustrates maintenance treatment regimens (Mono/Bi) over daily and monthly intervals across various studies.

FIG. 5C illustrates an overview of the maintenance and re-treatment regimens (e.g., T1, T2) for each regimen category.

### DETAILED DESCRIPTION OF SPECIFIC EMBODIMENTS

Although the following detailed description contains many specifics for the purpose of illustration, a person of ordinary skill in the art will appreciate that many variations and alterations to the following details are within the scope of the disclosure. Specific examples are used to illustrate particular embodiments; however, the disclosure described in the claims is not intended to be limited to only these examples, but rather includes the full scope of the attached claims. Accordingly, the following preferred embodiments of the disclosure are set forth without any loss of generality to, and without imposing limitations upon the claimed disclosure. Further, in the following detailed description of the preferred embodiments, reference is made to the accompanying drawings that form a part hereof, and in which are shown by way of illustration specific embodiments in which the disclosure may be practiced. It is understood that other embodiments may be utilized, and structural changes may be made without departing from the scope of the present disclosure. The embodiments shown in the Figures and described here may include features that are not included in all specific embodiments. A particular embodiment may include only a subset of all of the features described, or a particular embodiment may include all of the features described.

The leading digit(s) of reference numbers appearing in the Figures generally corresponds to the Figure number in which that component is first introduced, such that the same reference number is used throughout to refer to an identical component which appears in multiple Figures. Signals and connections may be referred to by the same reference number or label, and the actual meaning will be clear from its use in the context of the description.

As used herein, ‘treating’ or ‘treatment’ refers to one or more of: arresting the disease, reducing symptom severity, slowing progression. As used herein, the term “individual” is meant to include any human or non-human animal. The term “non-human animal” refers to all vertebrates, such as mammals and non-mammals, such as non-human primates, sheep, dogs, cats, horses, cattle, chickens, amphibians, reptiles, etc. Except where otherwise noted, “individual”, “patient”or “subject”are used interchangeably.

As used herein, “current density” refers to the current per unit area passing through a surface. Current density is a vector which may vary from point to point in a volume conductor. The “average current density”, unless otherwise stated, means the average current density magnitude over part of a specified surface, that is, a spatial average. Current density may also vary with time and the “time averaged current density” of a periodic waveform is simply the average over one period of the waveform of the average current density.

“Net DC” of a periodic waveform is the time-average of a current waveform over the period of the waveform.

The current density at an electrode having uniform current dispersion throughout the contact area may be calculated by dividing the applied current by the contact area.

As used herein, the term “monophasic pulse” may refer to a pulse having current flow in one polarity (either positive or negative) only.

As used herein, the term “biphasic pulse” may refer to a pulse formed by two contiguous phases of opposite polarity (e.g., a cathodic phase followed by an anodic phase). A biphasic pulse is considered balanced if the net charge across both phases is approximately zero, and unbalanced if the net positive and negative charges differ. In some embodiments, a biphasic pulse may include a brief interphase interval with zero current.

As used herein, the term “pulse width” may refer to the duration of a single pulse or phase, often specified in microseconds (μs) or milliseconds (ms). In a biphasic pulse, each polarity phase typically has its own pulse width.

As used herein, the term “duty cycle” may refer to the ratio (expressed as a percentage) of the active “on” time of a pulsed stimulation to the total cycle time. For example, in a repeating cycle of 1 second, if pulses are active for 0.5 seconds and inactive for 0.5 seconds, the duty cycle is 50%.

As used herein, the term “phase” may refer to a segment of a pulse or a defined sub-period of stimulation having a specified polarity, amplitude, and duration. In a biphasic pulse, each pulse contains two phases (one positive and one negative). A treatment session may also be subdivided into multiple phases, each delivering a different waveform type (e.g., first a monophasic phase, then a biphasic phase).

As used herein, the term “accumulated charge” may refer to the total electrical charge delivered over a specified period or during a particular portion (phase or subphase) of stimulation. Accumulated charge is typically measured in coulombs (C) and is relevant for assessing potential electrochemical effects at the electrode-tissue interface.

As used herein, the term “burst” may refer to a series of pulses delivered in rapid succession, often followed by a rest period before repeating. A burst can be characterized by parameters such as frequency (pulses per second), duration, amplitude, and duty cycle.

As used herein, the term “polarity” may refer to whether an electrode is positive (anodic) or negative (cathodic) relative to a reference during a given phase of stimulation. In monophasic stimulation, the polarity remains fixed during each pulse; in biphasic stimulation, it alternates within each pulse.

As used herein, the term “phosphene” may refer to a non-visual perception of light (e.g., a flash or spark) caused by electrical or mechanical stimulation of the retina or visual pathways, rather than by photons. Detection of phosphenes may indicate that stimulation is reaching the intended retinal layers.

As used herein, the term “therapy calendar” may refer to a data structure or schedule specifying the timing (e.g., dates, frequency), session lengths, and stimulation parameters (such as amplitude, frequency, and waveform type) for each treatment session over a defined therapy regimen.

Where numerical ranges or values are provided in this disclosure, they are intended to encompass each and every point within the stated range, as well as every value and subrange, whether or not explicitly recited. Thus, a range of “1 to 10” includes 1.0, 1.1, 2, 3.5, 9, 9.9, and 10.0, as well as all subranges (for example, 2 to 9 or 3.5 to 7.5). Unless otherwise specified, any stated range should be understood to include its endpoints and to disclose individual numerical values that fall within that range.

Wherever terms such as “approximately,” “about,” or “substantially” are used in conjunction with a numerical value, these terms indicate that the given numerical value is subject to a reasonable tolerance, as recognized by those skilled in the art—e.g., ±5%, ±10%, or any other practical variation. Specific examples of acceptable tolerance levels may vary depending on technical context, measuring equipment, or commercial practice.

Unless otherwise stated, percentages, ratios, proportions, and dimensions described herein are understood to fall within commonly acceptable engineering or scientific tolerances for the relevant field. Accordingly, such values should be interpreted so as to allow minor variations in accordance with normal design and manufacturing conditions.

Described herein are devices, systems, and methods for treating one or more conditions (such as AMD, geographic atrophy, etc.) by providing low-level microcurrent (μA) transpalpebral electrical stimulation to the eye. Specifically, the methods disclosed herein generally include applying patterned electrical stimulation to the ocular region transpalpebrally, where the patterned electrical stimulation is defined by a plurality of waveform parameters. The electrical stimulation may result in therapeutic effects such as improvement of disease indications such as AMD and geographic atrophy.

The retinal pigment epithelium (RPE) is a monolayer of cells that are linked by tight junctions forming a sheet which surrounds the neurosensory retina, separating it from the choroid. It therefore forms a spherical dome with the choroidal face being the basal side and the retinal face the apical. Differences in the rate of ion transport between the basal and apical membranes of RPE cells lead to a standing electrical potential difference across the layer, known as the Trans Epithelial Potential. The RPE is therefore electrically polarized with the retinal side more positive than the choroidal. Cell polarization is an important component of proper cellular function, especially for epithelial cells. Epithelial cells form layers which act as a barrier to prevent the passage of some molecules as well as modulate what components are found on both sides of the epithelial barrier. In the case of the RPE cell layer, it is important that these cells know which side of the barrier is the choroid (basolateral) and which side is the neural layer (apical) in order to ensure proper modulation of nutrients, ions and growth factors. It is believed that the polarity and magnitude of the transepithelial potential are important signals controlling aspects of cell function and disruption of this layer can result in pathological imbalance or reduction in expression of key molecules leading to cell death and ultimately vision loss.

The existence of the TEP relies on the ability of the RPE to create a charge imbalance between the basal and apical sides as well as the integrity of the cell junctions to maintain an electrically resistive barrier. The net positive charge in the apical extracellular space leads to a TEP of approximately 1.2-3.5 mV and is maintained by the tight junctions between the cells preventing the movement of ions down the gradient to restore equilibrium. The transepithelial electrical resistance (TEER) layer can reach 300-600 Ω·cm² across RPE cells.

Correct polarization provides orientation for the cell and directs the secretion of a wide range of biologically active proteins from RPE cells. RPE cells secrete vascular endothelial growth factor (VEGF) basally toward the choroid for healthy blood vessel maintenance. Pigment epithelial derived factor (PEDF), on the other hand, is normally secreted apically into the interphotoreceptor matrix (IPM) to promote photoreceptor/RPE/retinal health and reduce the formation of blood vessels in the neural layer. Accordingly, the side of the RPE layer where these proteins are secreted will impact the health and function of the retina and perturbations of this normal secretion program can lead to cellular degeneration/death and loss of visual acuity. As an example, oxidative stress, as seen in aging individuals, can alter the normal balance of VEGF and PEDF secretion from RPE cells, leading to reduced neuroprotection and increased potential for abnormal neovascularization. PEDF's neuroprotective activity has been reported in models of retinal degeneration, oxidative stress, ischemia, excessive light exposure, glutamate excitotoxicity, and other pathological conditions.

Muller cells (discussed below) are another source of PEDF within the nuclear retina. Muller cells are the primary resident glial cells of the retina that serve to detect infectious agents, mount appropriate immune and healing responses, and support and maintain the cells in the neural layer. Muller cells span nearly the entire retina and interact with each of the cells in the neural layer providing neurotrophic factors, removing metabolic waste and maintaining the composition of the extracellular environment, among other functions. A subset of Muller cells possesses unique stem cell characteristics that give them the ability to proliferate and transdifferentiate into other cell types and repopulate the neuronal cells of the retina; however, this regenerative potential is only realized naturally in zebrafish, birds and amphibians Preclinical models in vertebrates and mammals have shown the potential for Muller cells to be modulated to form photoreceptors and other retinal neuronal cells in vitro and in vivo, indicating that therapeutically unlocking their potential in humans might still be possible, though even in these models Muller cells did not seem to produce a meaningful number of mature cells.

One of the most important functions of Muller cells is to synthesize and secrete neurotrophic and neuroprotective factors to prevent neuronal cell death during disease and maintain healthy function. In addition to PEDF, other factors secreted by Muller cells include brain-derived neurotrophic factor (BDNF), basic fibroblast growth factor (bFGF), ciliary neurotrophic factor (CNTF), and insulin-like growth factor I (IGF-1). Many of these factors are found to be upregulated following injury to the retina in an attempt to prevent/slow neural retina degeneration. However, in chronic disease states, levels of these factors are found to be reduced in the aqueous humor of patients. Interestingly, RPE cells also secrete and express receptors for CTNF and CTNF treatment increases RPE cell survival in vitro, further highlighting that Muller cell and RPE cell synergy and cross-talk can mutually stimulate the health and function of the other cell population.

Noninvasive electrical stimulation of the eye has been reported as a promising therapy for either preserving or restoring vision in several retinal and optic nerve diseases. In particular, the 2016 review paper by Sehic, et al. provided a comprehensive review of the animal and clinical studies conducted up to that point, using a broad range of electrical stimulation parameters. Cao L, Liu J, Pu J, Milne G, Chen M, Xu H, et al. Polarized retinal pigment epithelium generates electrical signals that diminish with age and regulate retinal pathology. J Cell Mol Med. 2018 November; 22(11): 5552-64 showed that impressing a direct current (DC) electric field to mimic the natural TEP increased the expression of PEDF in RPE cells of CCL2/CX3CR1 double knockout (DKO) mice. It is noteworthy that when the polarity of the applied potential was reversed the effect vanished. It has been shown that low level electrical current could significantly promote the proliferation of mouse MCs in vitro and promote their neurogenic potentials (Enayati S, Chang K, Achour H, Cho K S, Xu F, Guo S, et al. Electrical Stimulation Induces Retinal Mëller Cell Proliferation and Their Progenitor Cell Potential. Cells. 2020; 9(3): 781). Non-invasive electrical stimulation stimulated proliferative MCs to migrate and transdifferentiate into a photoreceptor cell lineage. Electric stimulation can also improve photoreceptor survival and increased retinal functions in mice with inherited photoreceptor degeneration (Yu H, Enayati S, Chang K, Cho K, Lee S W, Talib M, et al. Noninvasive Electrical Stimulation Improves Photoreceptor Survival and Retinal Function in Mice with Inherited Photoreceptor Degeneration. Invest Ophthalmol Vis Sci. 2020 April 9; 61(4): 5).

The mechanism by which MCs were activated in this way is believed to be through voltage sensitive L-type calcium channels in the cell membrane because when a calcium channel blocker was used in cell cultures exposed to electrical stimulation the effect was abolished (Enayati et al., 2020). The waveforms were typically biphasic (Enayati et al., 2020, Yu et al., 2020).

Current electric stimulation treatment methods for treatment of AMD incur unacceptable side effects and/or provide only temporary relief of symptoms for the disease. The proposed disclosure describes methods, systems, and waveforms to treat AMD without causing unacceptable side effects.

### System

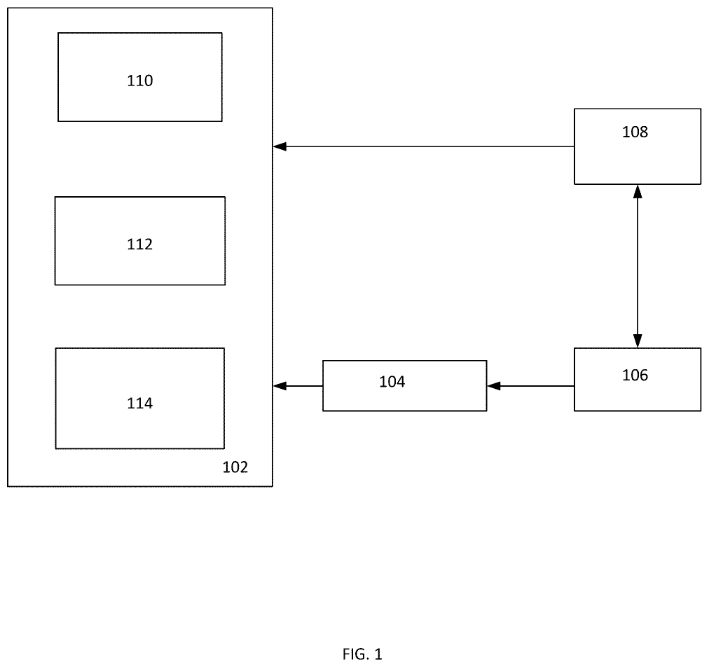

FIG. 1 illustrates a block diagram of an example system for generating electrical waveforms for delivery of electrical stimulation therapy to the eye for treatment of one or more conditions. As shown in FIG. 1, the system 100 includes an electrical signal source 102, and at least one electrode assembly 104. Optionally, the system may also include sensor 108. The arrangement 100 is shown in context operably coupled to eye-related tissue 106.

The electrical signal source 102 is a circuit or group of circuits that is configured to generate a controlled electrical signal. The at least one electrode assembly 104 is operably coupled to receive the electrical signal from the electrical signal source and configured to be attached to a portion of an eye or eye-related tissue 106 of a living being. The sensor 108 may be disposed proximate the eye-related tissue 106 and is configured to sense and generate one or more measurements for the eye-related tissue 106 (e.g., detection of pain, discomfort, phosphene generation, etc.).

In the embodiment of FIG. 1, the electrical signal source 102 comprises a controller 110, a stimulation circuit 112, and a power source 114. In this embodiment the controller 110 may suitably be a microcontroller that is configured to selectively cause the stimulation circuit 112 to generate (or not generate) the electrical stimulation signals. In general, to cause the stimulation circuit 112 to selectively provide or not provide stimulation signals, the controller 110 is operably coupled to provide control signals to the stimulation circuit 112. In some cases, the control signals further include signals that control the amplitude, pulse frequency, and/or pulse width of the stimulation signals generated by the stimulation circuit 112.

The stimulation circuit 112 is any now or hereafter known circuit that is configured to receive control signals from the controller 110 and generate electrical stimulation signals therefrom. In general, the stimulation circuit 112 produces stimulation signals in the form of electrical pulses in a pulse train, or pulse burst. As also discussed above, the pulse frequency, pulse width and amplitude may be varied. The stimulation circuit 112 may suitably be configured for manual adjustment of such values or automatic adjustment of the values via the controller 110. The stimulation circuit may be a constant current circuit and/or a constant voltage circuit.

It will be appreciated that a single integrated circuit may be employed as both the microcontroller 110 and stimulation circuit 112. In some embodiments, the microcontroller 110 provides the stimulation signal pulse train, which is then merely amplified by the stimulation circuit 112.

Referring again to FIG. 1, the one or more electrodes 104 are operably coupled to receive the stimulation signals from the stimulation circuit 112, and further configured to apply the signals to eye-related tissue. In at least some embodiments, the electrodes 104 are positioned in a transpalpebral manner. In various embodiments, the electrode assembly 104 may be the device as described in, for example, PCT Application PCT/US2022/050319 (published as WO 2023091611), filed on Nov. 17, 2022 by Masko et al., titled “METHOD AND SYSTEM FOR EYE TREATMENT,” incorporated herein by reference in its entirety. An example electrode assembly includes a plurality of electrodes to be positioned over the eyelid and a grounding electrode located at the base of the neck of an individual. Other electrode assemblies (such as those described in other applications discussed above and incorporated herein by reference) are within the scope of this disclosure.

The optimal size of the active electrode is ultimately a compromise between competing requirements. On one hand, a small electrode is preferred so that the current is confined to a small contact area as close as possible to the cornea. This minimizes dispersion of the current that could bypass the eye and ensures more of the current reaches the posterior sector of the retina. It also minimizes activation of the anterior retina. However, a small electrode leads to high current density when used in conjunction with a constant-current generator and this becomes more problematic with monophasic stimulation. Direct Current (DC) type stimulation can result in electrochemical reactions at the electrode site which can produce by-products that are harmful, alter local pH or drive unwanted ionized molecules into the skin by ionophoresis. For this reason, safety standards such as IEC 60601 stipulate DC currents as low as 10 μA. A DC current density at the electrode of less than 0.5 mA/cm² is considered safe for cathodic currents, with 1 mA/cm² being the corresponding limit for anodic current. In one or more embodiments of the present invention, an electrode contact area for an eye is configured to have large area such that the current is dispersed thereby reducing the average current density over the contact area. In one or more embodiments, the electrode contact area is configured to be shaped to match the aperture and not extend beyond the aperture of the eye. (Aperture, in this instance, means the anterior surface of the eyelid that lies within the bony orbit. The avoids the current bypassing the eye entirely through contacts that extend beyond the aperture of the eye. In one example, an electrode having an area of approximately 2 cm² has been found to be particularly suited to control the current dispersion using a pulsed constant current generator up to 1 mA. A higher current, up to 2 mA could also be used, in conjunction with a reduced duty cycle.

An electrode in direct contact with the cornea has advantages in that it is closer to the vitreous with less current like to escape around the eye. There are various types of electrodes including the DTZ type which comprises a fine flexible wire that is laid across the cornea and secured on either side of the eye with an adhesive pad. A gold foil electrode can also be used or a contact lens type electrode. In the latter case a coiled wire is embedded in the hydrogel of the lens and is exposed on the corneal face of the electrode such that when the lens is in situ a conductive connection to the cornea is achieved. The wire can be coiled in a spiral to increase the surface area of contact, thereby reducing the current density at surface of the cornea.

Direct application of currents to the cornea have been associated with adverse responses such as dry eye and irritation. It is important that in use the electrode continues to make a good contact with the eyelid, or cornea. A loss of contact area for any reason will result in an increase in current density for a constant current controlled generator. Preferably, the system can detect when the surface area of contact has reduced below an acceptable level and alert the user while reducing the current so as to manage the current density within acceptable limits.

The system is configured to maintain the current density at the cornea or contact (e.g., by controlling the applied voltage and determine the current flowing to the contact) at safe levels and, automatically detecting breach(es) of safe level(s) and in response alerting the user(and/or terminate or adjust the treatment). The system is configured to control the current density (e.g., at the contact of the eye) such that it does not exceed safe levels (e.g., using the waveforms discussed below). The system can include measurement features that determine current levels or current density (in real time) based on such measurements.

The return electrode should be as large as possible and located posterior to the retina so that current is directed towards the back of the eye through the retina. The large size helps with dispersion through the brain, as well as minimizing any skin irritation.

In some embodiments, the one or more active electrodes have a total area of conductive contact of between about 1.5 cm² and about 4 cm², and the one or more return electrodes have a conductive contact area that is at least twice that of the active electrode. This arrangement helps ensure safe current density levels at the active electrode, including a direct current (DC) density not exceeding approximately 0.5 mA/cm² and preferably less than 0.25 mA/cm². Additionally or alternatively, the controller can limit the time-integrated charge density at the active electrode to less than a predefined limit, thereby reducing the risk of tissue damage during extended stimulation.

The delivered charge density (CD) at an electrode can be calculated as follows:

$$C D = \{ ( net DC current in mA ) \times ( time in seconds ) \} \div ( conductive area of contact in {cm}^{2} ) .$$

For example, if a 1 mA monophasic pulse train at 50% duty cycle operates for a subphase duration of 60 seconds, the net charge delivered is 30 mC (millicoulombs). With a 1.5 cm² conductive area at the active electrode, the resulting charge density is 30 mC÷1.5 cm²=20 mC/cm², equivalent to 200 C/m². Charge densities up to about 480 C/m² have been used in transcranial DC applications; accordingly, in some embodiments a 480 C/m² limit could be applied. In some embodiments, more particularly, the predefined limit on the charge density during any given monophasic subphase is selected to be at or below about 200 C/m².

To further manage safety, the system can detect a potentially hazardous reduction in the contact area of the one or more active electrodes. For example, the controller may monitor an area-dependent electrical impedance parameter and reduce or halt stimulation if this parameter exceeds a preset acceptance threshold. The acceptance threshold can be determined during a calibration phase in which the electrodes are known to be fresh and fully contacting. Further accuracy can be gained by passing a known current between the active electrode and a third reference electrode of known contact area. The acceptance threshold can be set to a predefined margin above the measured baseline impedance.

All cells are electrically active, and the objective with therapeutic electrical stimulation is to alter the local electrical environment in the vicinity of target cell groups to achieve a beneficial response while minimizing adverse reactions. Typically, it is not the absolute magnitude of the electrode-applied current that matters, but rather the resulting electrical current density at the target cells and other tissues. Current density, rather than total current, determines both efficacy and potential risk of harm. Accordingly, management of current density is a key feature of this disclosure.

It is not only the magnitude of the current density that is significant. Current density is a vector having both magnitude and direction. The direction of current flow determines the locally generated electrical potential difference, including its polarity, which the target cells may detect.

The body is a volume conductor composed of tissues with widely differing electrical conductivities. The eye and periorbital area, as well as the head, present anatomically complex structures and tissues. Accordingly, an electrical current applied between two electrodes located on the head disperses in a three-dimensional flux that depends on local anatomy and tissue conductivity.

One objective of this disclosure is to stimulate specific retinal cell types, including Muller cells and the Retinal Pigment Epithelium (RPE). Using conventional technology, computer modeling was conducted to determine the current density throughout the eye when electrical stimulation is applied between a first electrode located at the front of the eye and a return electrode at the back of the head. More specifically, an MRI-based finite element model to estimate the dispersion of electric current in a volume conductor was performed. By means of this tool, the current density at the posterior portion of the retina (“retinal current density”)—when using waveforms, in particular monophasic pulses, and an electrode of about 2 cm² (contact area) providing a current density of about 500 μA/cm² at the electrode on one eye—is about 50 μA/cm² The modeling used a direct current of about 1 mA delivered through a contact area of approximately 2 cm² at the upper eyelid. Such modeling tools are available to those of ordinary skill in the art. The DC retinal current density due to a monophasic current pulse train of known duty cycle and amplitude can be scaled linearly from the modeling analysis.

The current density at various structures within the eye differs, reflecting variations in tissue conductivity. For example, the modeling determined that for a current of about 1 mA flowing through a contact area of approximately 2 cm² at the upper eyelid, the estimated average current density at the skin is about 300 μA/cm², at the cornea about 200 μA/cm², at the iris about 300 μA/cm², at the lens about 100 μA/cm², at the optic nerve about 50 μA/cm², at the optic disc about 100 μA/cm², at the contralateral eye about 6 μA/cm², and in the brain about 2 μA/cm². Those of ordinary skill in the art are familiar with using factors or multipliers to adjust the estimated values for different situations such as different pulse train parameters.

The vitreous is highly conductive and dominates current flow, being ultimately surrounded by less conductive bony structures.

The highest current density occurred at the active electrode.

With an electrode having a 2 cm² contact area positioned at the eyelid, and applying 1 mA continuous direct current, the average current density at the retina was estimated to be at about 50 μA/cm² based on the discussed modeling. A pulsed monophasic current at 50% duty cycle would halve that value, and further reductions would occur with currents less than 1 mA.

This electrode size, placement, and current amplitude produced safe current density levels at the eyelid, cornea, lens, iris, and other structures, even under monophasic stimulation.

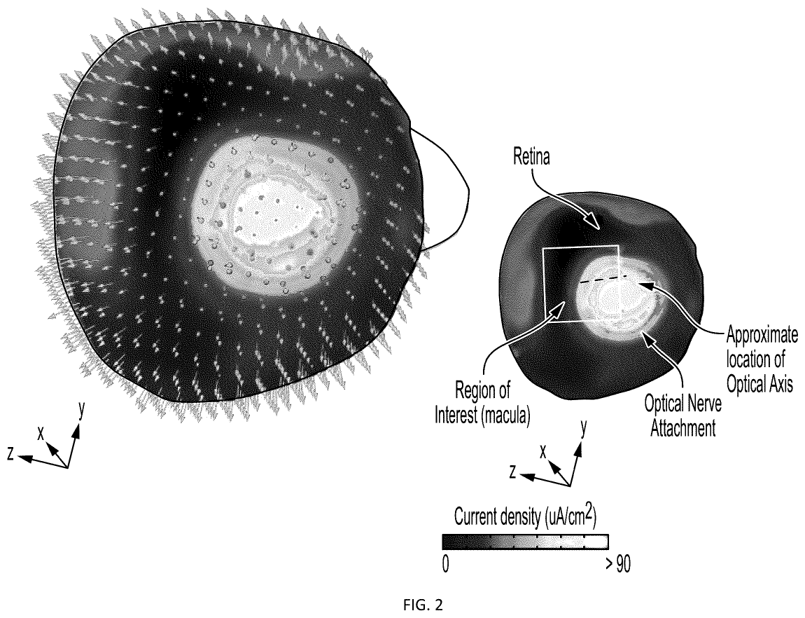

As shown in FIG. 2, the direction of the anodal current density vector at the eyelid was radially outward, rendering the inner retina more electrically positive relative to the choroid. In particular, the electrical potential across the RPE is enhanced by making the apical face more positive relative to the basal face. Previous findings indicate that the RPE transepithelial resistance typically ranges from about 200 to 600 Ω/cm²; thus, a current of 50 μA/cm² could yield about 10-30 mV of additional potential difference. A more common therapeutic current at 50% duty cycle and 500 μA amplitude would yield about 2.5-7.5 mV, close to the physiological range of 1.2-3.5 mV. A current of 200 μA and 50% duty cycle amplitude would still result in an average current density at the retina of 5 μA/cm² and a boost of 1.0 to 3 mV to the TEP. In AMD, the normal tight junctions between RPE cells—which provide high transepithelial resistance—are often compromised, leading to a partial collapse of the TEP. Externally applied current can help restore the TEP, even if the resistance drops below normal levels. It is estimated that the beneficial current density range at the retina is between 5 μA/cm² and 50 μA/cm². Also, as discussed below, studies performed by the Applicant have demonstrated the effectiveness of the retinal current density at or about this range.

It was concluded that this electrode arrangement simultaneously delivers a therapeutically relevant current density vector—both in magnitude and direction—at the retina, while constraining the current density at the active electrode to within recognized safety limits (approximately 0.5 mA/cm² for net DC currents).

In one or more embodiments, the system is configured to deliver adequate or sufficient treatment current to the retina and at the same sufficiently limit the current density at the active electrode or electrode contact area on the eyelid to provide the safe levels of operation. For instance, an active electrode that is too small delivering 1 mA, can give rise to a current density above 0.5 mA/cm² (previously discussed as being levels that breach a safe level). Likewise, an electrode (or set of electrodes) extending beyond the aperture of the eye diverts current around the eye, failing to generate the required current density at the retina. In one or more embodiments, the electrode or electrode area is adapted to have as large a contact area as possible in order to reduce current density for a given current, for example 1 mA, while not being so large as to extend beyond the aperture of the eye such a significant portion of the current goes around the eye and no through it.

Previously, it was believed that multiple small electrodes might deliver current to different parts of the retina more effectively. Applicant's research and analysis has discovered that using as large a conductive surface as practical at the active electrode to minimize current density locally while still directing a sufficient fraction of current to the retina provides better performance.

### Waveforms

The electrical stimulation waveforms described herein may be tailored for specific treatment regimens and/or specific patients. It should be appreciated that the waveforms described here may be delivered via a multi-polar, such as bipolar, tripolar, quad-polar, or higher-polar configuration or a monopolar configuration with distal return. The waveforms may be a sinusoidal, quasi-sinusoidal, square-wave, sawtooth, ramped, or triangular waveforms, truncated-versions thereof (e.g., where the waveform plateaus when a certain amplitude is reached), or the like.

As is described in more detail herein, when patterning of electrical stimulation waveforms is employed, waveform parameters such as the shape, the frequency, the amplitude, and the pulse width may be modulated. The frequency, pulse-width, and/or amplitude of the waveform may be modulated linearly, exponentially, as a sawtooth, a sinusoidal form, etc., or they may be modulated randomly. The stimulation can also be interrupted as part of the patterning. That is, the stimulation can be in an on/off condition, e.g., for durations of 1 second on/1 second off, 5 seconds on/5 seconds off, etc. Modulation of the waveform shape (e.g., rectangular vs. triangular vs. exponential) in a rhythmic or non-deterministic, non-rhythmic fashion may also be used. Thus, numerous variations in waveform patterning can be achieved. It should be understood that combinations of these parameter changed over time in a repetitive manner may also be considered patterning. In some instances, random patterning may be employed. Patterning may help to prevent patient habituation to the applied stimulation (i.e., may help to prevent the patient response to the stimulation decreasing during stimulation).

The stimulation may be delivered periodically at regular or irregular intervals. Stimulation bursts may be delivered periodically at regular or irregular intervals. The stimulation amplitude, pulse width, or frequency may be modified during the course of stimulation. For example, the stimulation amplitude may be ramped from a low amplitude to a higher amplitude over a period of time. In other variations, the stimulation amplitude may be ramped from a high amplitude to a lower amplitude over a period of time. The stimulation pulse width may also be ramped from a low pulse width to a higher pulse width over a period of time. The stimulation pulse width may be ramped from a high pulse width to a lower pulse width over a period of time. The ramp period may be between 1 second and 15 minutes. Alternatively, the ramp period may be between 5 seconds and 30 seconds.

The patterned stimulation waveforms described herein may be used to increase the comfort of the patient and/or may be used to improve the efficacy of the stimulation, and thus, described below are waveform parameters that may be used alone or in combination to increase comfort and/or efficacy.

It is also necessary to define the electrical stimulation waveform parameters, such as polarity, amplitude, waveshape and timing, which is to be applied to the electrode assembly in order to create the desired electrical conditions at the target cells. An objective of the present disclosure is to establish a sustained outflow of current across the posterior retina which has the effect of increasing the trans epithelial potential of the RPE. A sustained period may be defined as one in which the current reaches a steady state, albeit with a ripple component. This could occur within one tenth of a second but is likely to be more beneficial if sustained for longer such as 0.5 seconds, or 1 second. The longer the current can be safely and comfortably sustained the better.

While trying to set up the desired electrical conditions in one place, it is necessary to avoid electrical hazards, such as high current density at the skin or collateral stimulation to other sensitive organs. Therefore, there is a need for devices and techniques which establish the correct electrical connections to the transorbital area and apply the appropriate electrical energy to achieve a desired therapeutic advantage, while avoiding the creation of hazardous conditions.

In some instances, the waveform shape or modulation thereof may affect the comfort and/or efficacy of the stimulation. A continuous direct current where the active electrode is anodal and the return electrode is cathodal, could be used for a sustained period however this approach may create a hazard at the electrode sites where electrochemical changes can occur leading to tissue damage. Reversing the DC current flow for a sustained period to achieve an overall zero charge transfer may help to mitigate risk.

When the stimulator (electrode device) is configured to create a pulse-based electrical waveform, the pulses may be any suitable pulses (e.g., a square pulse, a haversine pulse, or the like). The pulses delivered by these waveforms may by biphasic, alternating monophasic, or monophasic, or the like.

A pulsed direct current, otherwise known as a monophasic pulse train, can be used. The duty cycle of the pulse train can be varied from 0 to 100%, in effect reducing the time-average current by the same proportion. The monophasic pulse train may have advantages over the DC current of the same average current by having a higher frequency content and embedded time periods where there is no current. Monophasic electrical stimulation presents particular problems due to electrochemical changes that occur at the electrodes with prolonged current in one direction. These changes can cause severe tissue irritation or burns at the electrode site. Safety standards limit the exposure to monophasic currents. While it may be desirable to achieve a sustained electric field at some target tissue remote from the stimulation electrode, it is necessary to manage the electrode DC current density to safe levels.

In some embodiments, the system provides stimulation comprising two or more phases, at least one of which delivers monophasic pulses and at least one of which delivers biphasic pulses. The amplitude of monophasic pulses can differ from the amplitude of biphasic pulses for therapeutic effect. A monophasic phase may be further divided into multiple subphases, each delivering exclusively positive or negative pulses, and each subphase can have a duration of about 1 second up to about 60 seconds. To manage safety, the total accumulated electrical charge in each subphase can be equalized or capped below a predefined threshold. In certain variations, the frequency of monophasic pulses ranges between about 20 Hz and about 40 Hz, while a biphasic phase may operate between about 15 Hz and about 25 Hz.

Furthermore, the controller may separate these phases with a rest interval in which no stimulation is provided, and the respective durations of the monophasic and biphasic phases may be unequal. A treatment session can thus deliver the monophasic phase first followed by the biphasic phase, or vice versa, with each phase lasting approximately 0.1 to 10 minutes (commonly around 1 minute). A treatment regimen may include multiple sessions in which the proportions of monophasic vs. biphasic phases vary according to a predefined schedule.

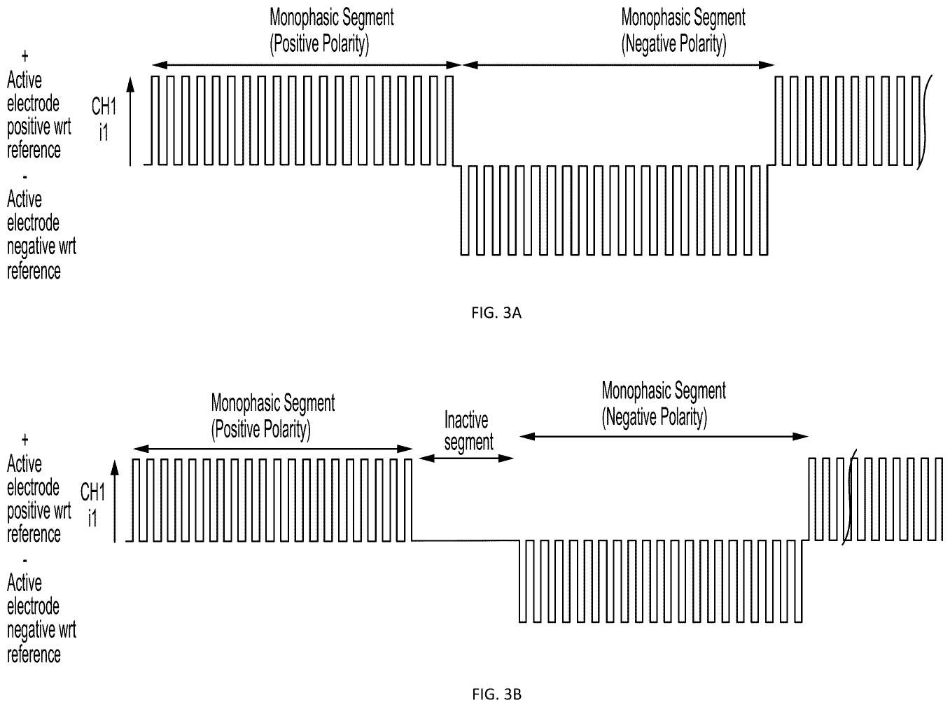

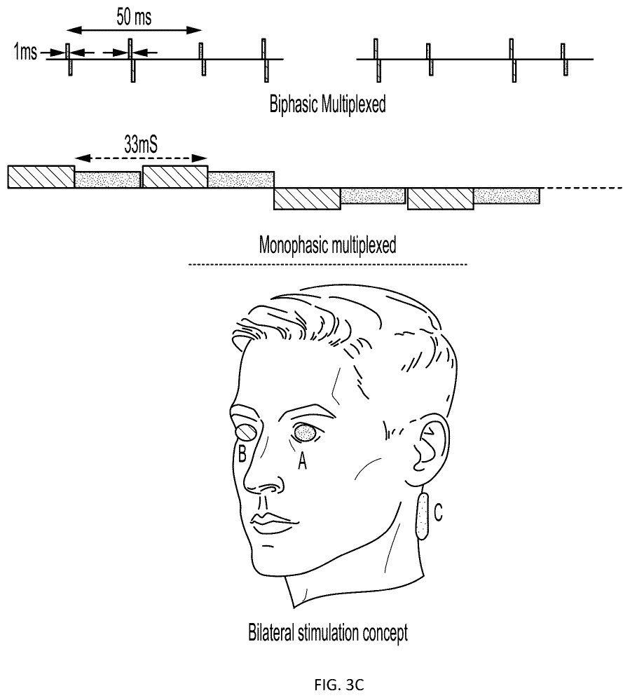

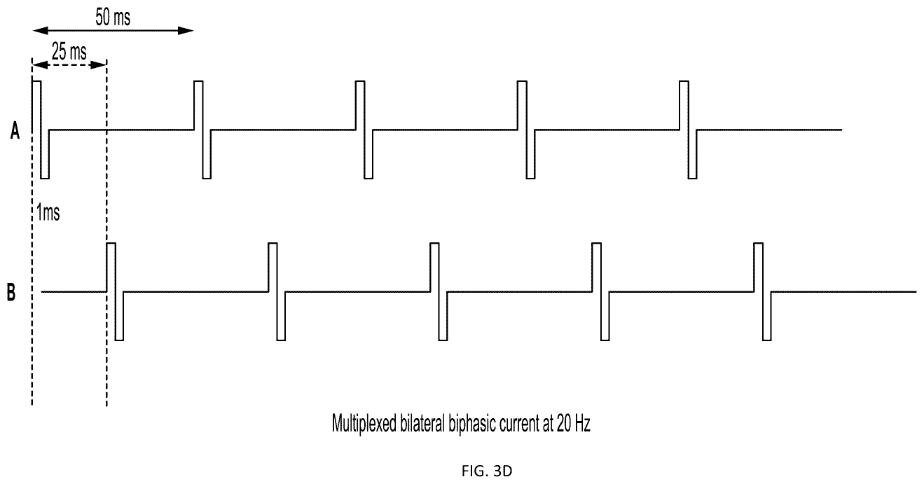

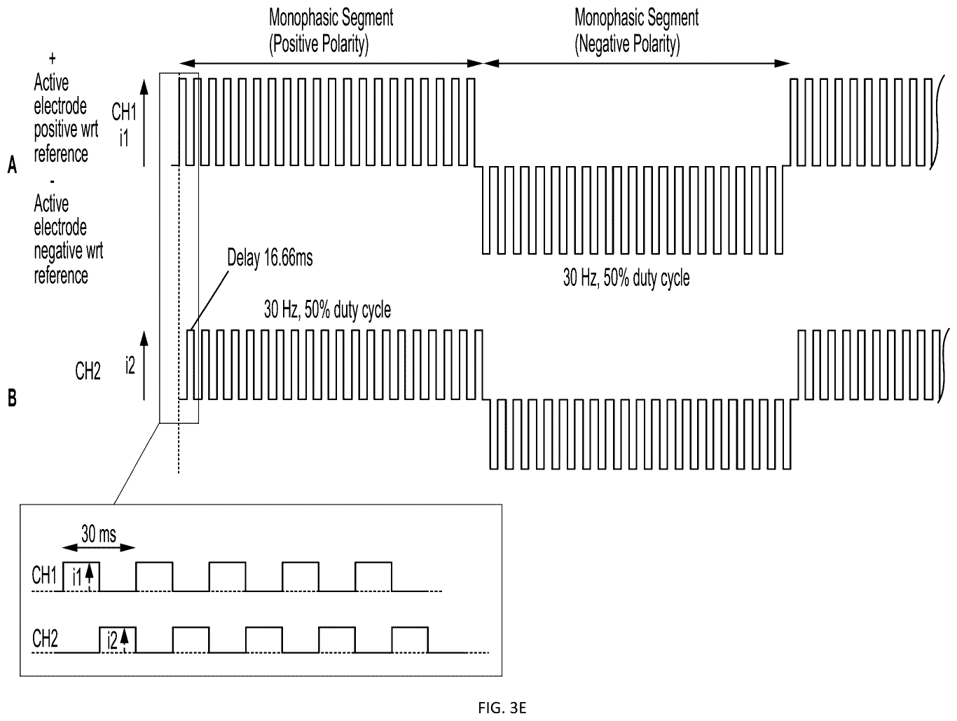

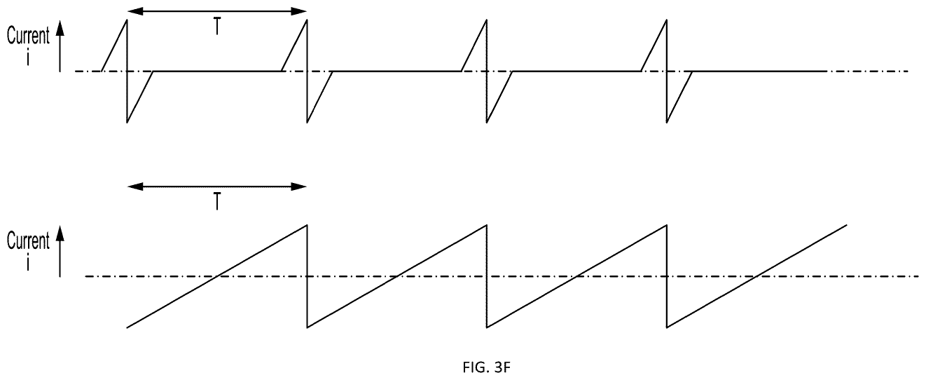

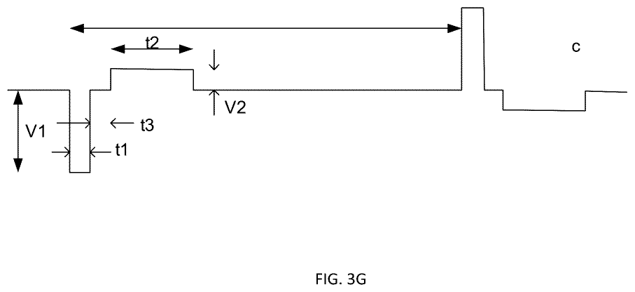

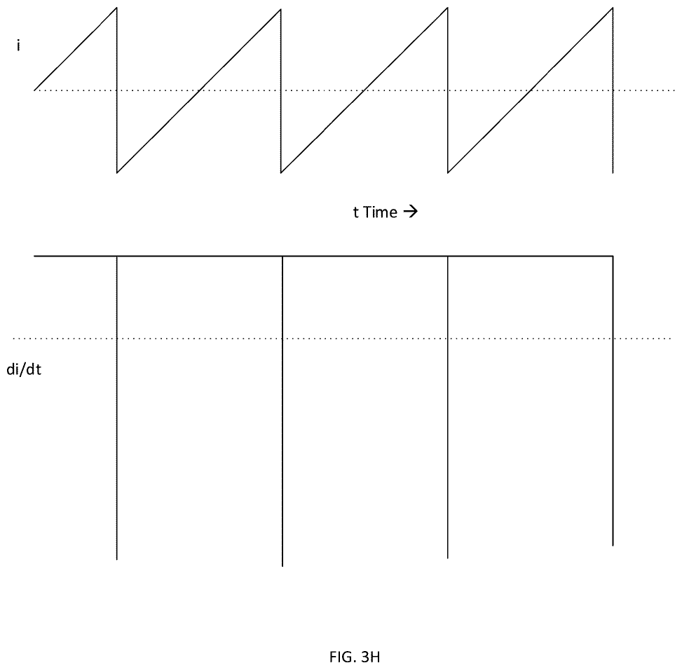

A monophasic segment comprising a sustained period at one polarity followed by a similar period of reverse polarity may mitigate risk due to monophasic stimulation (shown in FIG. 3A). The monophasic periods can be separated by a period of no stimulation. For example, a periodically reversing monophasic pulse train at about 25-35 Hz, about 25 Hz, about 30 Hz, about 35 Hz, having a 50% duty cycle may be used. The duration of the sustained monophasic segment may be about 0.5-2 seconds, about 0.5 s, about 1 s, about 2 s, although longer durations of 5 or 10 seconds are possible.

As previously discussed, monophasic currents present risks associated with the buildup of by-products of the electrochemical reactions taking place at the electrodes. In one or more embodiments, the system is configured to limit the time-accumulated current at any one polarity so the accumulation of harmful by products is thereby limited. In some cases, it is beneficial to apply a reverse polarity current for a period which partially reverses the build-up of electrochemical by-products (as shown in FIG. 3A). Preferably, the reverse polarity current is the same amplitude as the original current and maintained for the same time. In one embodiment there is a train of monophasic pulses for a first time period (e.g., about 1 s) immediately followed by a train of opposite polarity pulses at the same amplitude for one second. The period can be extended beyond one second, to 5, 10 or more seconds, but risks of adverse reactions would increase. In one embodiment the time accumulated current at any one polarity can be selected by the operator and the controller adjusts the pulse train durations accordingly.

It can also be beneficial to simply stop the pulse train for a period to allow the potentially harmful by-products disperse by diffusion into the tissue (shown in FIG. 3B). A combination of pulse trains of opposite polarity interspersed with rest period in which there are no pulses can be beneficial.

In one or more embodiments, the monophasic pulses may be multiplexed and applied bilaterally to both eyes (shown in FIG. 3C). A first train of monophasic pulses for a first time period may stimulate a first eye (while providing a charge-balancing phase to a second eye), and a second train of monophasic pulses for a second time period may stimulate the second eye. A reverse polarity current may then be applied to the first eye and the second eye respectively.

The controller can limit the time accumulated current below a desired threshold value by monitoring the output current (current passing through the electrode on the eye) and terminating or adjusting the current when it reaches the selected limit. A reverse polarity current could then be applied which could be the same magnitude and duration as the first pulse train. Alternatively, the reversed polarity pulse train could have different amplitude and duration but be arranged to achieve the same time accumulated charge such that charge balance between pulse trains is achieved.

When a pulse is biphasic, the pulse may include a pair of single-phase portions having opposite polarities (e.g., a first phase and a charge-balancing phase having an opposite polarity of the first phase). Each phase of the biphasic pulse may be either voltage-controlled or current-controlled. In some variations, both the first phase and the charge-balancing phase of the biphasic pulse may be current-controlled. In other variations, both the first phase and the charge-balancing phase of the biphasic pulse may be voltage-controlled. In still other variations, the first phase of the biphasic pulse may be current-controlled, and the second phase of the biphasic pulse may be voltage-controlled, or vice-versa. In some instances, a combination of current-controlled bilateral stimulation and voltage-controlled charge balancing may allow for unilateral stimulation, and by modifying the waveform shape, may allow for switching between areas of stimulation, e.g., between nostrils when electrodes are located in each nostril, as described herein.

Pulsed biphasic signals comprise two phases of opposite polarity within each pulse. These pulses typically achieve charge balance within a few milliseconds and are therefore less likely to cause harmful electrochemical effects at the electrodes. It may be desirable to mix periods of monophasic and biphasic stimulation within the same treatment session or between successive treatment sessions.

In some variations in which the waveform comprises a biphasic pulse, it may be desirable to configure the biphasic pulse to be charge-balanced, so that the net charge delivered by the biphasic pulse is approximately zero. In some variations, a biphasic pulse may be symmetric, such that the first phase and the charge-balancing phase have the same pulse width and amplitude. Having a symmetric biphasic pulse may allow the same type of stimulus to be delivered, e.g., to each eye. The pulses of a first phase may stimulate a first eye (while providing a charge-balancing phase to a second eye), while the pulses of the opposite phase may stimulate the second eye (while providing a charge-balancing phase to the first eye)-shown in FIG. 3D.

In other variations in which the waveform comprises a biphasic pulse, a biphasic pulse may be asymmetric, where the amplitude and/or pulse width of the first pulse may differ from that of the charge-balancing phase. Even if the biphasic pulse is asymmetric, the biphasic pulse may be charge-balanced. For example, the cathodic pulse may have lower amplitude but longer duration than the anodic pulse, or the cathodic pulse may have higher amplitude but shorter duration than the anodic pulse. In both instances, the charge injection (amplitude times duration) may be equal for each pulse, such that the net charge delivered by the biphasic pulse is approximately zero.

In some instances, the controller is configured to generate the stimulation waveform to minimize side effects and enhance efficacy. Of particular interest are waveforms where the rate of change of voltage or current with respect to time is biased in one direction. An example is a sawtooth waveform having a positive slope. (FIG. 3G). This has the advantage of having a net zero DC current and yet a longer period for which the rate of change of voltage across the retina is directed radially outward. The current through a capacitor is proportional to the rate of change of voltage across the capacitor. Cell membranes have capacitance and hence an alternating extracellular electric field with a biased rate of change, such as in the positive slope sawtooth, may be favorable for stimulation of such membranes. The linear ramp of the sawtooth could be replaced by an increasing sinusoidal phase or any monotonically increasing waveform that biases the duration for which the rate of change in the outward direction is longer than the inward direction.

Note that the rate of change of the voltage may be in the opposite direction to the voltage. The voltage on the vitreous may be less than the choroid, but if the rate of change of voltage across the retina is positive then current will flow outward through capacitive channels. In other words, the current is out of phase with the voltage.

Mixed pulse trains: Monophasic pulse trains of the appropriate current density, polarity, frequency and phase charge, may have special significance for the RPE cells, and biphasic pulse trains may be more suited to stimulating Muller cells, the system can be configured to apply microcurrent stimulation that that applies (defined by) both waveforms in succession may be more beneficial than treating with each separately. The two waveforms can be combined in various ways.

The systems described herein may be configured to generate one of more waveforms at frequencies suitable for stimulating targeted tissue (e.g., a nerve). The frequency may affect the comfort and/or efficacy of the stimulation. Generally, the frequency is preferably between about 5 Hz and about 100 Hz. In some of these variations, the frequency is preferably between about 10 Hz and about 90 Hz. In some of these variations, the frequency is preferably between about 30 Hz and about 70 Hz. In others of these variations, the frequency is preferably between about 40 Hz and about 60 Hz. In others of these variations, the frequency is preferably between about 15 Hz and about 30 Hz. In some variations, the frequency may be about 5 Hz, about 10 Hz, about 15 Hz, about 20 Hz, about 25 Hz, about 30 Hz, about 35 Hz, about 40 Hz, about 45 Hz, about 50 Hz, etc.

The maximum amplitude or modulation thereof may affect the comfort and/or efficacy of the stimulation (less than about 1000 μA). In some variations, the amplitude may be variable and may also depend on whether the pulse is current controlled or voltage-controlled. When a stimulator is configured to deliver a pulse-based waveform, in some variations, the amplitude of the pulses may be constant over time. In other variations, the amplitude of the pulses may vary over time. This may reduce patient accommodation. In some variations, the amplitude of pulses may increase (linearly, exponentially, etc.) from a minimum value to a maximum value, drop to the minimum value, and repeat as necessary. In some variations, the amplitude of the pulses may vary according to a sinusoidal profile. In another variation, the amplitude may periodically increase from a baseline amplitude to a higher amplitude for a single pulse. In yet another variation, the amplitude of the pulses may follow a periodically increasing and decreasing pattern between two lower amplitudes, and periodically increase to a higher amplitude for a single pulse or for a plurality of pulses (e.g., two pulses). In yet another variation, a higher amplitude pulse (or pulses) may be preceded by a brief pause (i.e., no current delivery). Each of these types of amplitude modulation may be implemented alone or in combination with any other type of amplitude modulation. In some variations in which the amplitude varies over time, the amplitude may vary at a frequency suitable for reducing patient accommodation or increasing patient comfort.

The waveform parameters (e.g., frequencies, pulse widths, and amplitudes) may be changed to preferentially activate the tissue. For example, FIGS. 3A-3H illustrate exemplary waveforms configured to preferentially activate tissue with a substantially net zero charge accumulation. In some instances, the waveforms described herein may be delivered in a continuous fashion, while in other instances, the waveforms may be delivered in a non-continuous fashion having on periods and off periods, which may reduce patient discomfort. Exemplary on/off durations include without limitation, 1 second on/1 second off, 1 second on/2 seconds off, 2 seconds on/1 seconds off, 5 seconds on/5 seconds off, 0.2 seconds on/0.8 seconds off, less than 1 second on/less than 10 seconds off.

The waveforms may be optimized for various patients. A waveform may be assessed to determine if it is a patient-optimized waveform by delivering an electrical stimulus comprising the waveform to the patient using the system described herein. The method may comprise first delivering a waveform at the lowest amplitude and/or pulse width and asking the patient for feedback on the sensation as the amplitude and/or pulse width is increased. The method may then comprise assessing whether the patient feels any sensation during delivery of the electrical stimulus (e.g., detection of phosphene by the patient) and/or based on sensor data relating to eye activity or patient discomfort. In some embodiments, the patient report of phosphenes is used as an indicator that the electrical stimulus is reaching the target retinal tissues. As used herein, “phosphenes” are impressions of light (e.g., visual flashes) that occur in the eye in the absence of incoming light, resulting from stimulation of the retina (e.g., stimulation of specific retinal cells, such as bipolar cells). In some embodiments, the efficacy in treating retinal diseases (e.g., macular degeneration) to restore or maintain vision is integrally linked to delivering a level of ocular stimulation (e.g., electrical stimulation) that induces the visible appearance of phosphenes when a patient's eyes are closed. If not, a different waveform may be selected (e.g., having a different combination of parameters, such as frequency, amplitude, pulse width, on/off period, or the temporal modulation of these parameters). The method may further comprise ensuring that the patient is not experiencing discomfort. If the patient is experiencing discomfort, the method may be restarted with a new waveform, or the amplitude and/or the pulse width may be reduced to alleviate discomfort. Similarly, the method may comprise ensuring that the sensation during application of the waveform is comfortable to the patient. The amplitude and/or pulse width may be adjusted to achieve patient comfort. Comfort may be assessed with the patient's eyes both open and closed.

A waveform may be designated as a patient-optimized waveform if the patient perceives the waveform as the most comfortable and/or effective waveform felt that day; and/or if the patient perceives phosphenes or other visual percepts. In each case of an identified patient-optimized waveform, a lower or slightly higher amplitude and/or pulse width may be tested to determine whether the same sensation can be achieved using a lower amplitude and/or pulse width and/or if discomfort is observed.

### Treatment Regimen

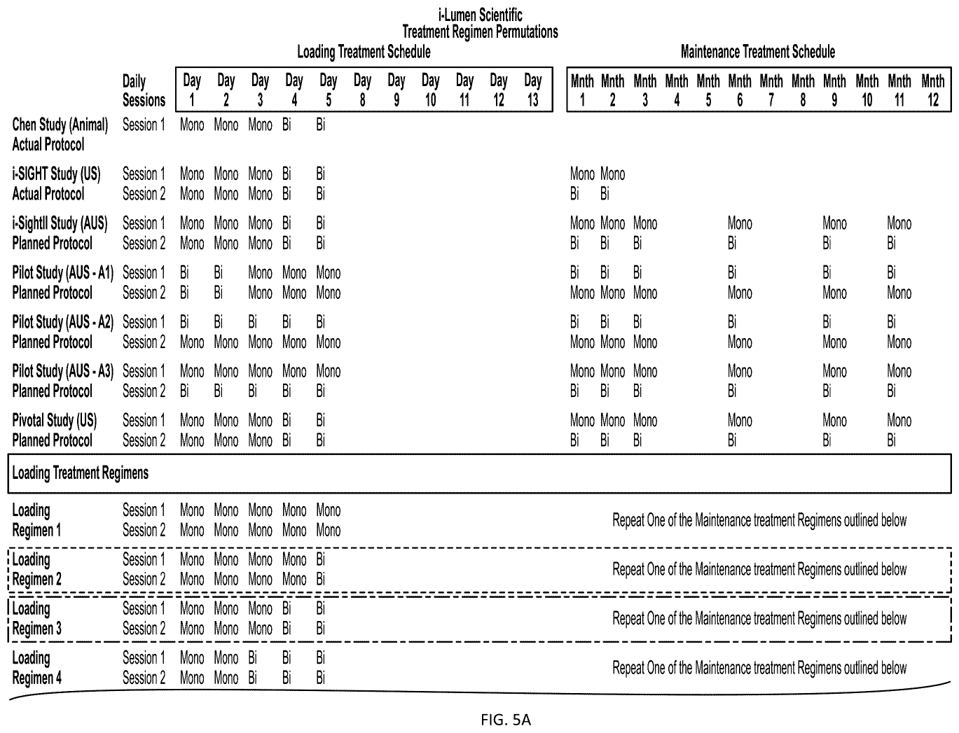

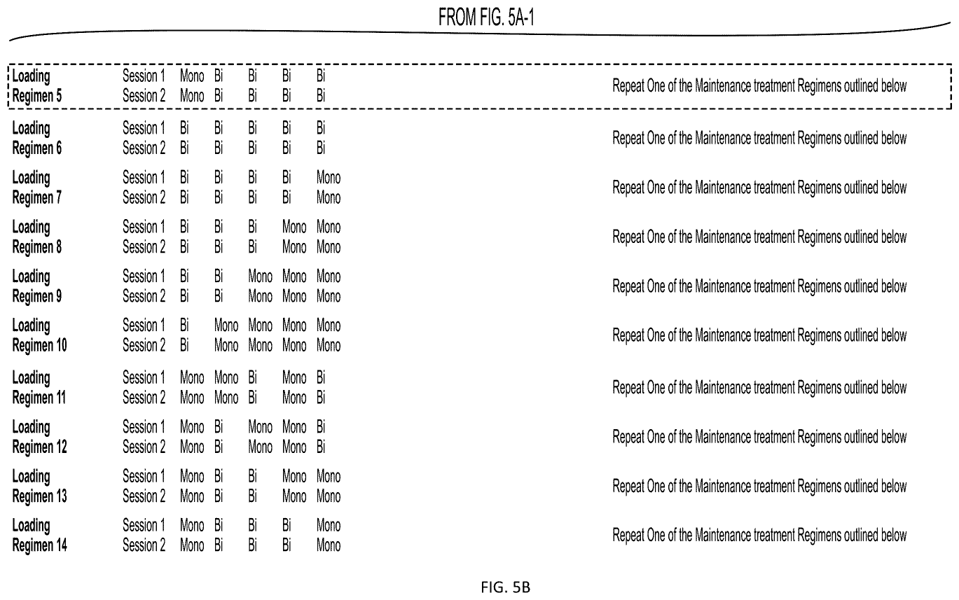

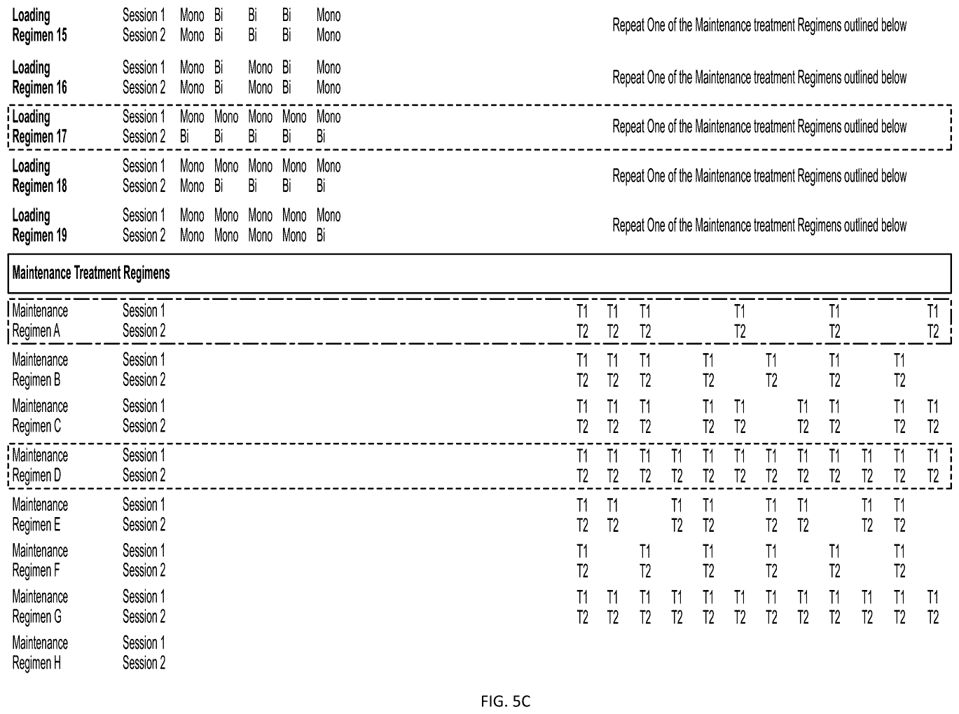

A treatment regimen can include one or more loading treatment sessions and one or more maintenance treatment sessions. The following parameters of a treatment regimen may be optimized to achieve a desired treatment outcome: (i) frequency of treatment days (e.g., about 4-12 days for loading treatment sessions and monthly treatments (e.g., every one month after 1 year, every 3 months after second year, etc.) during the maintenance treatment sessions; (ii) number of treatment sessions per day (e.g., about 1, 2, 3, 4, etc. treatment sessions per day); (iii) stimulation types (e.g., monophasic, biphasic, combined monophasic/biphasic, etc.); (iv) treatment session time period (e.g., about 2-60 mins with one or more rest periods). Example treatment sessions are provided in FIGS. 5A, 5B and 5C. However, other treatment sessions are within the scope of this disclosure. For example, one or more of the above parameters may be adjusted based on an individual.

To produce improvements in AMD or other indications, one or more of the above waveform patterns/phases may be used to provide electrical stimulation for a predetermined time period to the patient's eyes during regular (e.g. daily, weekly, or monthly) treatment sessions—loading followed by maintenance.

A treatment session can include delivery of a waveform including monophasic and/or biphasic pulse trains for a predetermined amount of time. For example, a treatment session may include two phases, wherein the first phase lasts a first time period with monophasic stimulation followed by a second phase for a second time period of biphasic stimulation (or vice versa), that is repeated for a treatment time. In another example, a treatment session may include equal alternating periods of monophasic and biphasic stimulation without or without a rest period between the monophasic and biphasic pulses repeated for a period of several minutes (e.g., about 16 minutes).

In another example, a treatment session can be established where a patient is treated for several days with one waveform (e.g., monophasic) and then with the alternative waveform (e.g., alternating monophasic/biphasic) on subsequent days. For example, a patient might have 2 treatments of monophasic per day for two days followed by two days in the which 2 treatments of biphasic are applied each day. In another embodiment one treatment each day would be monophasic while the other would be biphasic (e.g., for 4 days). A further embodiment would be where every treatment comprises a monophasic and biphasic phase. For example, a treatment might comprise 24 minutes where 12 minutes are monophasic, and 12 minutes are biphasic.

In some embodiments, the controller is further configured to implement a therapy regimen spanning multiple sessions, each session being exclusively monophasic, exclusively biphasic, or a combination thereof. A “therapy calendar” can be stored in the controller's memory, specifying the treatment schedule (e.g., dates, frequency, whether unilateral or bilateral treatment) and the stimulation parameters for each phase. The data structure may include a patient identifier, a total number of planned sessions, and specific session dates. For each session, the system may record or specify session duration, the number of phases, whether each phase is monophasic or biphasic, and parameters such as frequency, amplitude, pulse width, burst length, and polarity.

In various embodiments, a treatment session may be bilateral (i.e., treatment of both eyes at the same time during a single treatment session) or unilateral (i.e., treatment of one eye during a single treatment session).

Examples of bilateral treatment can include, for example, an alternating or sequenced stimulation pattern where a first eye receives a stimulation pattern for a first time period followed by second time period of ‘rest’ (no stimulation). The first time period may or may not be equal to the second time period. During the rest period for the first eye—i.e., the second time period, the second eye a stimulation pattern followed by a similar rest period (where the first eye again receives stimulation during the second eye's rest period). The stimulation delivered to each eye could be monophasic (eye 1)/monophasic (eye 2), biphasic (eye 1)/biphasic (eye 2), or some combination of monophasic (eye 1)/biphasic (eye 2) or monophasic (eye 1)/monophasic (eye 2).

In another example bilateral stimulation session, the first eye may receive monophasic stimulation while the second eye is receiving biphasic stimulation—i.e., the stimulation would be delivered simultaneously to both eyes. After a predetermined time, the stimulation is swapped between eyes so that the first eye receives biphasic stimulation, and the second eye receives monophasic stimulation. Optionally, booth eyes may receive the same stimulation type-monophasic or biphasic throughout the treatment session. Here, the simulation delivered to both eyes could be monophasic for a specified period of time, followed by a rest period and subsequently a biphasic stimulation for a specified period of time (or vice versa).

In some variations, the patterned electrical stimulation is applied by a system (e.g., system of FIG. 1) comprising a plurality of patterned stimulation waveforms stored in memory. In some of these variations, the applied patterned stimulation is randomly selected from the plurality of stored patterned stimulation waveforms in accordance with a treatment regimen. In some of these variations, the plurality of stored patterned stimulation waveforms are patient-optimized waveforms. In some variations, the applied patterned stimulation is stored in memory as a patient-optimized waveform.

In certain embodiments, the system further comprises a switching mechanism operably connected to the controller and configured to direct stimulation pulses bilaterally. Each eye may receive its own pulse train with defined polarity, amplitude, frequency, or duty cycle, and pulses can be time-multiplexed so that one eye receives stimulation while the other rests. Alternatively, bursts of pulses may alternate between the two eyes, or each eye may receive different waveform modes (e.g., one eye monophasic, the other biphasic) and/or different frequencies, amplitudes, or pulse widths.

In some examples, the devices and systems disclosed herein are suited for use in conjunction with exogenous and/or endogenous stem cell transplantation therapies. For example, a method may comprise delivery of electrical stimulation before, during, or after stem cell transplantation to improve cell survival, repair and/or replacement. In illustrative examples, the use of methods and systems disclosed herein may enhance native cell survival, transplanted cell survival, transplanted cell integration, and functional synapse formation and/or axon regeneration. Non-limiting examples of endogenous stem cell types which may be suitable for transplantation in combination with systems or devices of the present disclosure include Miiller cells, retinal pigment epithelial cells (RPE cells) and ciliary pigmented epithelial cells (CPE). Non-limiting examples of exogenous stem cells suitable for transplantation according to some embodiments of the disclosure include neural stem cells (NSC's), mesenchymal stem cells (MSCs) derived from bone marrow, adipose tissue or dental pulp and stem cells from the inner cell mass of the blastocyst and induced pluripotent stem cells (iPSCs). See, for example, “Using Electrical Stimulation to Enhance the Efficacy of Cell Transplantation Therapies for Neurodegenerative Retinal Diseases: Concepts, Challenges, and Future Perspectives”, Abby Leigh Manthey, et al., Cell Transplantation, Vol. 26, pp. 949-965, 2017.

In further embodiments, the controller can generate a periodic pulse train that achieves net zero DC current by employing asymmetric waveforms. For example, positive and negative rectangular, exponential, or triangular pulses of equal area but different duration can be used, or a sawtooth shape where the positive slope extends longer than the negative slope. Such asymmetric approaches maintain overall charge balance while potentially creating a beneficial bias for retinal cell stimulation. Moreover, the controller may periodically vary one or more parameters (e.g., amplitude, pulse width, frequency) in a ramped or randomized manner over time to reduce patient habituation.

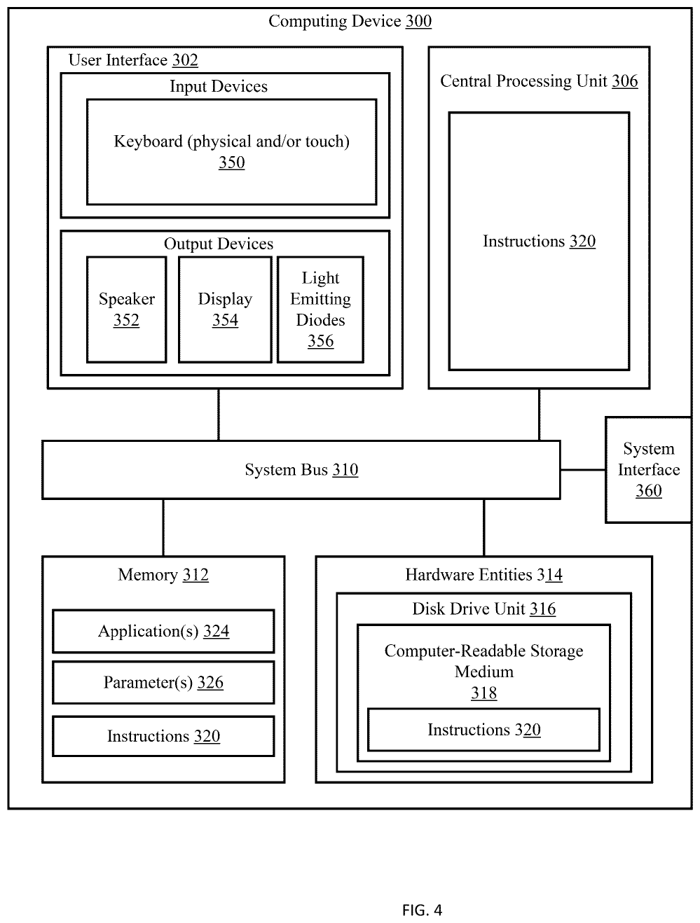

Referring now to FIG. 4, there is provided an illustration of an architecture for a computing device 300. The electrical signal source 102 of FIG. 1 is/are the same as or similar to computing device 300. As such, the discussion of computing device 300 is sufficient for understanding electrical signal source 102 and/or other components of FIG. 1.

Computing device 300 may include more or less components than those shown in FIG. 4. However, the components shown are sufficient to disclose an illustrative solution implementing the present solution. The hardware architecture of FIG. 4 represents one implementation of a representative computing device, as described herein. As such, the computing device 300 of FIG. 4 implements at least a portion of the method(s) described herein.

Some or all components of the computing device 300 can be implemented as hardware, software and/or a combination of hardware and software. The hardware includes, but is not limited to, one or more electronic circuits. The electronic circuits can include, but are not limited to, passive components (e.g., resistors and capacitors) and/or active components (e.g., amplifiers and/or microprocessors). The passive and/or active components can be adapted to, arranged to and/or programmed to perform one or more of the methodologies, procedures, or functions described herein.

As shown in FIG. 4, the computing device 300 comprises a user interface 302, a Central Processing Unit (CPU) 306, a system bus 310, a memory 312 connected to and accessible by other portions of computing device 300 through system bus 310, a system interface 360, and hardware entities 314 connected to system bus 310. The user interface can include input devices and output devices, which facilitate user-software interactions for controlling operations of the computing device 300. The input devices include, but are not limited to, a physical and/or touch keyboard 350. The input devices can be connected to the computing device 300 via a wired or wireless connection (e.g., a Bluetooth® connection). The output devices include, but are not limited to, a speaker 352, a display 354, and/or light emitting diodes 356. System interface 360 is configured to facilitate wired or wireless communications to and from external devices (e.g., network nodes such as access points, etc.).

At least some of the hardware entities 314 perform actions involving access to and use of memory 312, which can be a Random Access Memory (RAM), a disk drive, flash memory, a Compact Disc Read Only Memory (CD-ROM) and/or another hardware device that is capable of storing instructions and data. Hardware entities 314 can include a disk drive unit 316 comprising a computer-readable storage medium 318 on which is stored one or more sets of instructions 320 (e.g., software code) configured to implement one or more of the methodologies, procedures, or functions described herein. The instructions 320 can also reside, completely or at least partially, within the memory 312 and/or within the CPU 306 during execution thereof by the computing device 300. The memory 312 and the CPU 306 also can constitute machine-readable media. The term “machine-readable media”, as used here, refers to a single medium or multiple media (e.g., a centralized or distributed database, and/or associated caches and servers) that store the one or more sets of instructions 320. The term “machine-readable media”, as used here, also refers to any medium that is capable of storing, encoding or carrying a set of instructions 320 for execution by the computing device 300 and that cause the computing device 300 to perform any one or more of the methodologies of the present disclosure.

To further clarify, sequence(s) and/or temporal order of steps of various processes or methods that are described herein are illustrative and should not be interpreted as being restrictive. Accordingly, it should be understood that although steps of various processes or methods or connections or sequence of operations may be shown and described as being in a sequence or temporal order, they are not necessarily limited to being carried out in any particular sequence or order.

It should be understood that claims that include fewer limitations, broader claims, such as claims without requiring a certain feature or process step in the appended claim or in the specification, clarifications to the claim elements, different combinations, and alternative implementations based on the specification, or different uses, are also contemplated by the embodiments of the present invention.

It should be understood that combinations of described features or steps are contemplated even if they are not described directly together or not in the same context.

Clinical research studies performed, and ongoing, by the Applicant on patients diagnosed with AMD have demonstrated the effectiveness of the treatment. In a randomized controlled study, the system was configured to deliver both monophasic and biphasic pulse currents (up to 1 mA) to an individual eye. Specifically, 30 Hz monophasic pulse trains were used at a 50% duty cycle with 1-second polarity-reversal intervals, and 20 Hz biphasic pulse trains with a 5 ms pulse width. Each treatment session lasted 16 minutes, during which the active electrode, having a surface area of about 2 cm², was placed on the patient's eyelid, and the return electrode was placed on the back of the neck.

Under these conditions, the average current delivered was about 662 μA for the 50%-duty-cycle monophasic phase and about 692 μA for the biphasic phase (range 550-1000 μA). Based on this, the typical DC current density at the retina was approximately 17 μA/cm², and the corresponding DC current density at the electrode was about 331 μA/cm². Participants underwent an initial 5-day loading regimen (two sessions per day), consisting of three days of monophasic stimulation followed by two days of biphasic stimulation. They then completed two additional maintenance treatment days—one day at Month 1 and one day at Month 2—each day consisting of one monophasic session and one biphasic session. By the Month 3 timepoint, 50% of treated participants achieved or exceeded a clinically meaningful improvement in best-corrected visual acuity of more than eight ETDRS letters.

In general, a benefit of the embodiments of the present invention is that treatment is non-invasive. It involves the described systems and processes and does not require in order to be productive or accomplish the desired stimulation, invasive procedures such as injections into the eye or around the eye. In general, the effectiveness does not involve or require the patient to be given medication to enhance or effectuate the discussed microcurrent stimulation processes to be effective. It can essentially be applied on its own if desired to treat AMD (or other eye conditions) in patients.

The system is configured in one or embodiments to determine in real time the current (or similar characteristic) flowing through the contact to the eye under the treatment and adjust the voltage or other setting to accomplish the predefined parameter for the treatment. The controller is configured to use measurement to adjust the current and/or voltage in real time based on the measurement to accomplish the predefined treatment parameters.

As discussed, one or more embodiments include a 2 cm² contact area. In some embodiments this contact (or contact area) is a single distinct contiguous surface (“single piece”) and this provides certain advantages in manufacturing and use. In some embodiments a contact (or contact area) is made of multiple distinct contiguous surfaces that positioned together to operate to apply the stimulation (e.g., such as multiple pieces placed closed next to each) to have the same approximate shape or size as the 2 cm² electrode. In other words, in some embodiments a single distinct contiguous electrode has advantages and, in some embodiments, having the electrode not be contiguous has advantages (such as flexibility). In addition, in some embodiments, the objective is to apply a voltage or current at the eyelid or on the cornea of the eye under the application to have the net DC current density at the electrode to tissue interface to be between 50 and 500 μA/cm² and the current density at the retina of that eye is to be between 5 μA/cm² and 50 μA/cm². This can be performed by for example using two distinct distanced electrical contacts on the eye lid of an eye, where each contact is contiguous, and the system controller controls the current to each contact to meet the stated parameters. In this case the current density at each contact would be within these limits.

It is to be understood that the above description is intended to be illustrative, and not restrictive. Although numerous characteristics and advantages of various embodiments as described herein have been set forth in the foregoing description, together with details of the structure and function of various embodiments, many other embodiments and changes to details will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein,” respectively. Moreover, the terms “first,” “second,” and “third,” etc., are used merely as labels, and are not intended to impose numerical requirements on their objects.

Electrode configurations and placements are designed to ensure safe current density levels and minimize adverse reactions. Safety mechanisms may be included to monitor impedance, contact area, and current limits to prevent excessive charge accumulation. Charge-balancing techniques, such as biphasic pulses or alternating monophasic pulses, may be implemented to mitigate risks of electrochemical by-products at the electrode interface.

Electrical stimulation parameters, including amplitude, frequency, and pulse width, may be optimized based on patient-specific factors. The system may employ a combination of monophasic and biphasic pulses, with varying duty cycles and rest periods to enhance efficacy and patient comfort. Patterned stimulation may be used to prevent habituation and maintain therapeutic benefits over time. Treatments may be administered according to predefined schedules, including loading and maintenance phases.

The examples provided in this disclosure are for illustrative purposes and are not exhaustive. Various modifications, improvements, and adaptations may be made without departing from the underlying principles. Any references cited herein are incorporated by reference to the extent permitted by applicable law. Unless otherwise indicated, singular terms include their plural equivalents, and vice versa.

What is claimed is:

1. A system for delivering electrical stimulation to at least one eye of a patient, comprising:
one or more active electrodes configured to be positioned near the anterior part of the eye;
one or more return electrodes configured to be positioned on the patient; and
a controller operably coupled to the one or more active electrodes and the one or more return electrodes, the controller being configured to generate electrical stimulation signals having a defined amplitude, current density, polarity, frequency, and duty cycle,
wherein the electrical stimulation signals including anodal monophasic pulses are delivered transpalpebrally or via direct corneal contact via the one or more active electrodes having a total area of conductive contact of between about 1.5 cm² and about 4 cm².

2. The system of claim 1, wherein a stimulation comprises two or more phases, at least one of which delivers monophasic pulses and at least one of which delivers biphasic pulses, and wherein the amplitude of the monophasic pulses is different from the amplitude of the biphasic pulses.

3. The system of claim 2, wherein a monophasic phase is divided into a plurality of subphases, each subphase delivering a train of pulses having exclusively positive polarity or exclusively negative polarity.

4. The system of claim 3, wherein the subphases are arranged to deliver an equal accumulated electrical charge and/or to limit the total accumulated electrical charge in each subphase to less than a predefined threshold.

5. The system of claim 3, wherein each subphase has a duration of at least about 1 second, optionally about 5 seconds, 10 seconds, 30 seconds, or 60 seconds.

6. The system of claim 2, wherein an accumulated charge in at least the monophasic phase is controlled by adjusting either the amplitude or the pulse width of the pulses.

7. The system of claim 2, wherein the frequency of the monophasic pulses is between about 20 Hz and about 40 Hz.

8. The system of claim 2, wherein the amplitudes of the monophasic and biphasic phases are determined based on the patient's tolerance threshold for each mode and are automatically managed thereafter.

9. The system of claim 2, wherein the frequency of the biphasic phase is in the range of about 15 Hz to about 25 Hz.

10. The system of claim 2, wherein the two or more phases are separated by a rest phase in which no stimulation is provided.

11. The system of claim 2, wherein the duration of a phase, whether monophasic, biphasic, or rest, is in the range of about 0.1 to 10 minutes, preferably about 1 minute.

12. The system of claim 2, wherein a treatment session comprises delivering the monophasic phase and the biphasic phase in either order.

13. The system of claim 2, wherein the respective durations of the monophasic and biphasic phases are unequal.

14. The system of claim 2, wherein a treatment regimen comprises multiple treatment sessions in which the proportions of the monophasic and biphasic phases are varied according to a defined schedule.

15. The system of claim 2, wherein the controller is further configured to implement a therapy regimen for the patient that spans multiple treatment sessions, each session being exclusively monophasic, exclusively biphasic, or a combination of monophasic and biphasic pulses, the regimen being defined by a therapy calendar specifying dates and the stimulation parameters for each session.

16. The system of claim 15, wherein the therapy calendar is defined by a data structure that includes a patient identifier, a total number of treatment sessions, and dates of treatment.

17. The system of claim 16, wherein, for each treatment session, the data structure further specifies whether the eye treatment is unilateral, sequential bilateral, or simultaneous bilateral.

18. The system of claim 17, wherein, for each eye treated in each session, the data structure specifies a session duration and a pulse train description including a number of phases.

19. The system of claim 18, wherein, for each phase of the session, the data structure specifies whether the phase is monophasic or biphasic, a frequency, an amplitude, a pulse width, a burst length, and a polarity.

20. The system of claim 1, wherein the one or more active electrodes are located near the anterior part of the eye, and the one or more return electrodes are located posterior to the eye, with the active electrode not extending beyond the aperture of the eye.

21. The system of claim 1, wherein the one or more return electrodes have a conductive contact area that is at least twice that of the one or more active electrodes.

22. The system of claim 1, wherein the controller is configured to limit the stimulation signal so that the direct current (DC) density at the one or more active electrodes does not exceed about 0.25 mA/cm².

23. The system of claim 1, wherein the controller is configured to limit the time-integrated charge density at the one or more active electrodes to less than about 200 C/m².

24. The system of claim 1, wherein the controller includes a mechanism to detect a potentially hazardous reduction in the contact area of the one or more active electrodes.

25. The system of claim 24, wherein the mechanism comprises detecting an increase in an area-dependent electrical impedance parameter beyond an acceptance threshold.

26. The system of claim 25, wherein the acceptance threshold is determined during a calibration phase in which the one or more active electrodes are known to be fresh and fully contacting the patient and to be a predefined amount higher than the parameter measured during the calibration phase.

27. The system of claim 1, further comprising a switching mechanism operably connected to the controller and configured to direct stimulation pulses bilaterally to both eyes of a patient, such that each eye receives its own pulse train having defined polarity, amplitude, frequency, or duty cycle.

28. The system of claim 27, wherein pulses are time-multiplexed to both eyes.

29. The system of claim 27, wherein the controller delivers bursts of pulses alternately to each eye such that, while one eye is receiving pulses, the other eye is resting.

30. The system of claim 27, wherein the two eyes can receive differing stimulation parameters, including any one or more of monophasic vs. biphasic waveforms, amplitude, frequency, or pulse width.

31. The system of claim 1, wherein the controller is configured to generate a periodic pulse train having net zero DC current, and wherein positive and negative parts of said pulse train are asymmetric in amplitude or duration while maintaining overall charge balance.

32. The system of claim 31, wherein the asymmetric waveform comprises positive and negative rectangular, exponential, or triangular pulses of equal area but that differ in duration.

33. The system of claim 31, wherein the asymmetric waveform comprises a sawtooth shape in which a positive slope extends for a longer duration than a negative slope.

34. The system of claim 33, wherein the positive slope refers to a positive rate of change of current entering the one or more active electrodes.

35. The system of claim 1, wherein the controller is configured to periodically vary at least one stimulation parameter selected from amplitude, pulse width, or frequency in a ramped or randomized manner over time, thereby reducing patient habituation during ocular stimulation.

36. The system of claim 1, wherein when an average DC current density at the one or more active electrodes is between 50 and 500 μA/cm², an average DC current density at a posterior portion of a retina of the eye is 5 to 50 μA/cm².
